(19) United States
(12) Patent Application Publication
(10) Pub. No.: US 2013/0053337 A1
(43) Pub. Date: Feb. 28, 2013

(54) GUANIDINYLATED AMINOGLYCOSIDE-LIPID CONJUGATES

(71) Applicants: Frank Schweizer, Winnipeg (CA); Smritilekha Bera, Gandhimagars (IN); George Zhanel, Winnipeg (CA)

(72) Inventors: Frank Schweizer, Winnipeg (CA); Smritilekha Bera, Gandhimagars (IN); George Zhanel, Winnipeg (CA)

(73) Assignee: UNIVERSITY OF MANITOBA, Winnipeg, MB (CA)

(21) Appl. No.: 13/582,823

(22) PCT Filed: Mar. 4, 2011
(86) PCT No.: PCT/IB2011/002534

§ 371 Date: Nov. 14, 2012

Related U.S. Application Data
(60) Provisional application No. 61/311,074, filed on Mar. 5, 2010.

(51) Int. Cl. C07H 15/22; A61P 31/04; A61K 31/7036

(52) U.S. Cl. 514/41; 536/13.8; 536/13.2; 514/40

(57) ABSTRACT

Guanadinylated aminoglycoside-lipid conjugates are prepared. Such conjugates may comprise an aminoglycoside such as an aminoglyucoside antibiotic like neomycin or kanamycin, at least one guanidino group attached to a primary or secondary carbon atom of the aminoglyucoside group and at least one lipid group attached through a bond or a linker to a branched carbon atom of the aminoglycoside. These conjugates exhibit improve antibacterial activity and may be used in conjunction with another antibiotic.

### BACKGROUND OF THE INVENTION

I. Field of the Invention

The present invention relates generally to antimicrobial drug development. More particularly, it concerns the therapeutic potential of cationic aminoglycoside-lipid conjugates as antimicrobial agents.

II. Description of Related Art

In recent years, the explosive growth of multidrug resistant bacteria in hospitals and the community have led to an emerging crisis where an increasing number of antibiotics cease to be clinically effective. The various mechanisms by which resistance to antibiotics develops has prompted interest in novel therapeutics with novel modes of action that are able to prevent or delay the emergence of resistance (Dzidic et al., 2008). One such class of currently studied antibacterial agents are cationic amphiphiles comprised of polycationic lipids (Bera et al., 2008), cationic peptide antibiotics (Hancock and Sahl, 2006), cationic lipopeptides (Makovitski et al., 2006), synthetic mimics of cationic peptide antibiotics (Scott et al., 2008), and cationic polymers (Kügler et al., 2005). Although several modes of action have been proposed, most researchers agree that the antibacterial effect of cationic amphiphiles involves disruption of the bacterial envelope induced by removal or substitution of divalent positively charged counterions by alkylammonium ions (Gilbert and Moore, 2005). This mode of action has been shown to limit the risk of cross-resistance (Hancock and Sahl, 2006).

Over the years, cationic lipids formulated as cationic liposomes have found applications as drug delivery systems against infectious diseases and gene transfection. Moreover, cationic lipids including benzalkonium chlorides, sphingosine and fatty amines, chlorhexidine, cationic polymers are known to exhibit broad-spectrum antibacterial activities. Many of these agents have been in use as antiseptics and disinfectants for several decades with little or no occurrence of resistance (Gilbert and Moore, 2005). However, there remains a need for novel classes of antibacterial agents with reduced resistance.

### SUMMARY OF THE INVENTION

The present invention addresses the needs discussed above by providing novel aminoglycoside-lipid conjugates that exhibit antibacterial activity. In some aspects, the aminoglycoside-lipid conjugates are guanidinylated. Indeed, some of the aminoglycoside-lipid conjugates discussed herein show marked improvements over traditional aminoglycoside antibiotics, such as neomycin and kanamycin. Additionally, methods of treating bacterial infections using these aminoglycoside-lipid conjugates are also provided by the present invention.

Accordingly, in some aspects, the present invention provides a guanadinylated-aminoglycoside-lipid conjugate comprising (a) an aminoglycoside group; (b) at least one guanidino group attached to a primary or secondary carbon atom of the aminoglycoside group; (c) at least one lipid group attached through a bond or a linker to a branched carbon atom of the aminoglycoside; or a salt thereof. In certain embodiments, the aminoglycoside-lipid conjugate comprises at least two guanidino groups attached to a primary or secondary carbon atom of the aminoglycoside group.

In another embodiment, the present invention provides an aminoglycoside-lipid conjugate comprising at least one guanidino group attached to a primary or secondary carbon atom of the aminoglycoside, wherein at least one lipid is conjugated at a primary hydroxy position of the aminoglycoside through a linker. In certain embodiments, the aminoglycoside-lipid conjugate comprises at least two guanidino groups attached to a primary or secondary carbon atom of the aminoglycoside.

The term “glycoside” refers to a compound in which a sugar group is bound to a non-carbohydrate moiety. Typically the sugar group (glycone) is bonded through its anomeric carbon to another group (aglycone) via a glycosidic bond that has an oxygen, nitrogen or sulfur atom as a linker. A “simple sugar,” or monosaccharide, is the basic structural unit of carbohydrates, which cannot be readily hydrolyzed into simpler units. The elementary formula of a simple monosaccharide is CₙH₂ₙOₙ, where the integer n is at least 3 and rarely greater than 7. Simple monosachharides may be named generically according on the number of carbon atoms n: trioses, tetroses, pentoses, hexoses, etc. Simple sugars may be open chain (acyclic), cyclic or mixtures thereof. In these cyclic forms, the ring usually has 5 or 6 atoms. These forms are called furanoses and pyranoses, respectively. The ‘D-’ and ‘L-’ prefixes may be used to distinguish two particular stereoisomers which are mirror-images of each other. The term simple sugar also covers O-acetyl derivatives thereof. An “amino sugar” refers to a derivative of a sugar, deoxy sugar, sugar acid or sugar alcohol, where one or more hydroxy group(s) has been replace with one more amino group(s). A “simple amino sugar” refers to a derivative of a simple sugar, simply deoxy sugar, simply sugar acid or sugar alcohol, where one or more hydroxy group(s) has been replace with one more amino group(s). These terms also cover N- and O-acetyl derivatives thereof.

A guanidine functional group has the general structure (NH)C(NH₂)₂. In some embodiments, the guanidino group is —NHC(NH)NH₂.

Lipids or lipid groups may be conjugated to an aminoglycoside and are described herein. In some embodiments, “lipid,” “lipid moiety,” or “lipid group” (used interchangeably) refers to a straight-chain hydrocarbon radical having 5 carbons or higher, wherein the radical may comprise single, double, and/or triple bonds or may be cyclic or aromatic. In certain embodiments, the straight-chain hydrocarbon radical has between 5 and 45 carbon atoms. In certain embodiments, a lipid may comprise only single bonds. In certain embodiments, a lipid may comprise 20 or fewer double bonds. In certain embodiments, a lipid may comprise at most or at least 19, 18, 17, 16, 15, 14, 13, 12, 11, 10, 9, 8, 7, 6, 5, 4, 3, 2, or 1 double bond(s), or any range derivable therein. In certain embodiments, a lipid may comprise 10 or fewer triple bonds. In certain embodiments, a lipid may comprise at most or at least 9, 8, 7, 6, 5, 4, 3, 2, or 1 triple bond(s), or any range derivable therein. In certain embodiments, a lipid may be of the formula CₐH₂ₐ₊₁, wherein a is 5-45. In certain embodiments, a is at least or at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or higher, or any range derivable therein. In certain embodiments, one or more of the hydrocarbon chain(s) of the lipid is alkyl_{(C5-45)}, for example, a straight-chain alkyl_{(C5-45)}. In other embodiments, one or more of the hydrocarbon chains is an alkenyl group. Non-limiting examples of lipids include —C₅H₁₁, —C₁₁H₂₃, —C₁₅H₃₁, —C₁₉H₃₉ and —C₁₇H₃₁. In still further embodiments, the lipid may be cyclic or aromatic. In certain embodiments, the lipid may be defined as:

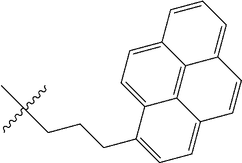 [SMILES: *CCCc1ccc2ccc3cccc4ccc1c2c34]

An aminoglycoside-lipid conjugate may comprise more than one lipid.

The linker may be, for example, an amide linker, an aminoacyl linker, an ester linker, an oxyacyl linker, a thioester linker, an oxythioacyl linker, a carbamate linker, a urea linker, a thiourea linker, an ether linker, a carbonate linker, a triazole linker, or an amino linker. In particular embodiments, the conjugation is through an amide linker. As used herein, the words “link,” “linkage,” “linker,” or “bound” refer to covalent binding between species, unless specifically noted otherwise. An “amide linker” refers to the following connection, each shown here attached to a lipid group: —NHC(O)-lipid. An “aminoacyl linker” refers to —C(O)NH-lipid. An “ester linker” refers to —OC(O)-lipid. An “oxyacyl linker” refers to —C(O)O-lipid. A “thioester linker” refers to —OC(S)-lipid. An “oxythioacyl linker” refers to —C(S)O-lipid. A “carbamate linker” refers to aminoglycoside-NHC(O)O-lipid. A “urea linker” refers to aminoglycoside-NHC(O)NH-lipid. A “thiourea linker” refers to aminoglycoside-NHC(S)NH-lipid. An “ether linker” refers to —O—. A “carbonate linker” refers to —OC(O)O—. An “amino linker” refers to —NH-lipid. Any of these linkers may be used with any aminoglycoside-lipid conjugate described herein, unless specifically noted otherwise. It is further noted that “through a linker” is to mean through that linker alone and no other atoms are comprised in the linker. In certain embodiments, a connection between an aminoglycoside and a lipid “comprises” any of these linkers—this means that other atoms may be found in the linkage. For example, the following linker comprises an amide linker: —NHC(O)—CH(CH₃)-lipid.

In any embodiments herein that employs an aminoglycoside, the aminoglycoside may be an aminoglycoside antibiotic. Such agents are well known in the art. In certain embodiments, the aminoglycoside antibiotic is selected from the group consisting of a neomycin, a kanamycin, paromomycin, amikacin, a gentamicin, netilmycin, a streptomycin, tobramycin, a hygromycin and a spectinomycin. In some embodiments, the aminoglycoside antibiotic is selected from the group consisting of a neomycin, kanamycin, amikacin, streptomycin, tobramycin and hygromycin. In more particular embodiments, the aminoglycoside antibiotic is neomycin or kanamycin. Any one or more of these aminoglycoside antibiotics may be excluded, in certain embodiments.

Any aminoglycoside employed herein, in isolation or conjugated to a hydrophobe, may exist with one or more free amino groups (—NH₂). In such embodiments, the aminoglycoside or aminoglycoside-hydrophobe conjugate may exist as a trifluoroacetic acid salt (TFA salt). Any aminoglycoside discussed herein may comprise a guanidino group (—NH—C(═NR)NHR, wherein R is H or an amino protecting group) attached to a primary or secondary carbon atom of the aminoglycoside, and the aminoglycoside may be isolated or may be conjugated to a hydrophobe. In certain embodiments, then, an aminoglycoside of the aminoglycoside-hydrophobe comprises at least one primary or secondary amino group, and the aminoglycoside-hydrophobe is present as a trifluoroacetic acid salt.

Non-limiting examples of aminoglycoside-lipid conjugates of the present invention include:

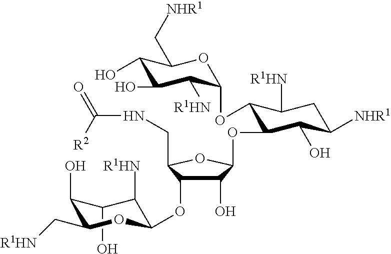

wherein R₁═C(NH)NH₂; and R₂═C₅H₁₁, C₁₁H₂₃, C₁₅H₃₁, C₁₉H₃₉, C₁₇H₃₁,

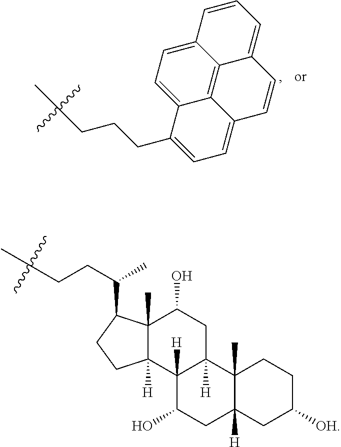

Additional non-limiting examples of aminoglycoside-lipid conjugates of the present invention include:

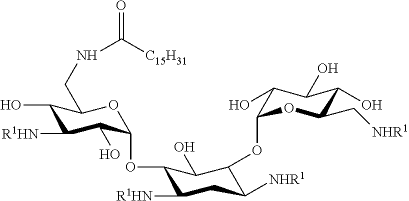

wherein R₁═C(NH)NH₂.

The present invention also contemplates pharmaceutical compositions. A pharmaceutical composition may comprise, for example, an aminoglycoside-lipid conjugate comprising at least one guanidino group attached to a primary or secondary carbon atom of the aminoglycoside in a pharmaceutically acceptable formulation. The aminoglycoside-lipid conjugate may be any aminoglycoside-lipid conjugate described herein.

The aminoglycoside-lipid conjugates as disclosed herein may be used in methods of treatment. In some aspects, the invention provides a method of treating a bacterial infection in a subject comprising administering to the subject an effective amount of an aminoglycoside antibiotic-lipid conjugate comprising at least one guanidino group attached to a primary or secondary carbon atom of the aminoglycoside. The aminoglycoside-lipid conjugate may be any aminoglycoside-lipid conjugate described herein. The bacteria causing the bacterial infection may be a multi-drug resistant bacteria, for example.

The bacterial infection may be caused by, for example, a Gram-positive bacteria. Non-limiting examples of Gram-positive bacteria include Staphylococcus aureus methicillin-resistant Staphylococcus aureus (MRSA), Staphylococcus epidermidis, methicillin-resistant S. epidermidis (MRSE), Enterococcus faecalis, Enterococcus faecium, or Streptococcus pneumoniae.

The bacterial infection may be caused by, for example, a Gram-negative bacteria. Non-limiting examples of Gram-negative bacteria include E. coli, gentamicin-resistant E. coli or amikacin-resistant E. coli, P. aeruginosa or gentamicin-resistant P. aeruginosa.

In certain embodiments regarding treatment of a bacterial infection using a aminoglycoside-lipid conjugate of the present invention, the minimum inhibitory concentration of the aminoglycoside antibiotic-hydrophobe conjugate (MIC) is ≦64 μg/mL. In certain embodiments, the MIC is ≦32 μg/mL. In certain embodiments, the minimum inhibitory concentration is about, at most about, or at least about 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or lower μg/mL, or any range derivable therein. Certain methods contemplate an additional step comprising administration of a second antibacterial agent.

In certain embodiments, methods of the present invention may further comprise diagnosing a subject as needing treatment for a bacterial infection prior to administering an aminoglycoside antibiotic-hydrophobe conjugate comprising at least one guanidino group attached to a primary or secondary carbon atom of the aminoglycoside. In other embodiments, methods of the present invention may further comprise administering treatment to a subject who has been identified as needing treatment for a bacterial infection.

Also contemplated are methods of preventing a bacterial infection in a subject comprising administering to the subject an effective amount of an aminoglycoside antibiotic-lipid conjugate comprising at least one guanidino group attached to a primary or secondary carbon atom of the aminoglycoside. Such methods may further comprise diagnosing the subject as needing preventative treatment for the bacterial infection prior to administering the aminoglycoside antibiotic-hydrophobe conjugate.

Another general aspect of the present invention contemplates a method of treating a bacterial infection in a subject comprising administering to the subject an effective amount of a neomycin-lipid conjugate comprising at least one guanidino group attached to a primary or secondary carbon atom of the aminoglycoside, wherein the effective amount of the neomycin-lipid conjugate is less than the effective amount of neomycin.

Another general aspect of the present invention contemplates a method of treating a bacterial infection in a subject comprising administering to the subject an effective amount of a kanamycin-lipid conjugate comprising at least one guanidino group attached to a primary or secondary carbon atom of the aminoglycoside, wherein the effective amount of the kanamycin-lipid conjugate is less than the effective amount of kanamycin.

Embodiments discussed in the context of methods and/or compositions of the invention may be employed with respect to any other method or composition described herein. Thus, an embodiment pertaining to one method or composition may be applied to other methods and compositions of the invention as well.

As used herein, “conjugation,” “conjugate,” and “attach” or “attached” refer to covalent bonds between entities, unless specifically noted otherwise.

When used in the context of a chemical group, “hydrogen” means —H; “hydroxy” means —OH; “oxo” means ═O; “halo” means independently —F, —Cl, —Br or —I; “amino” means —NH₂; “hydroxyamino” means —NHOH; “nitro” means —NO₂; imino means ═NH; “cyano” means —CN; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; “thio” means ═S; “thioether” means —S—; “sulfonamido” means —NHS(O)₂—; “sulfonyl” means —S(O)₂— (see below for definitions of groups containing the term sulfonyl, e.g., alkylsulfonyl); “sulfinyl” means —S(O)—; and “silyl” means —SiH₃.

For the groups below, the following parenthetical subscripts further define the groups as follows: “(Cn)” defines the exact number (n) of carbon atoms in the group. “(C≦n)” defines the maximum number (n) of carbon atoms that can be in the group, with the minimum number of carbon atoms in such at least one, but otherwise as small as possible for the group in question, e.g., it is understood that the minimum number of carbon atoms in the group “alkenyl_{(C≦8)}” is two. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3 to 10 carbon atoms). (Cn-n′) defines both the minimum (n) and maximum number (n′) of carbon atoms in the group. Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3 to 10 carbon atoms)).

The term “alkyl” when used without the “substituted” modifier refers to a non-aromatic monovalent group with a saturated carbon atom as the point of attachment, a linear or branched, cyclo, cyclic or acyclic structure, no carbon-carbon double or triple bonds, and no atoms other than carbon and hydrogen. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “substituted alkyl” refers to a non-aromatic monovalent group with a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “alkenyl” when used without the “substituted” modifier refers to a monovalent group with a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “substituted alkenyl” refers to a monovalent group with a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and —CH═CHBr, are non-limiting examples of substituted alkenyl groups.

As used herein, “protecting group” refers to a moiety attached to a functional group to prevent an otherwise unwanted reaction of that functional group. The term “functional group” generally refers to how persons of skill in the art classify chemically reactive groups. Examples of functional groups include hydroxyl, amine, sulfhydryl, amide, carboxyl, carbonyl, guanidino, etc. Protecting groups are well-known to those of skill in the art. Non-limiting exemplary protecting groups fall into categories such as hydroxy protecting groups, amino protecting groups, sulfhydryl protecting groups and carbonyl protecting groups. Such protecting groups, including examples of their installation and removal, may be found in Greene and Wuts, 1999, incorporated herein by reference in its entirety. Triazole aminoglycoside-(amino acid)ₙ conjugates described herein are contemplated as protected by one or more protecting groups—that is, the present invention contemplates such conjugates in their “protected form.” Non-limiting examples of carboxylic acid protecting groups include benzyl (Bn) and t-butyl. Non-limiting examples of amino protecting groups include Bn, carbobenzyloxy (Cbz), t-butoxycarbonyl (Boc) and 9-fluorenylmethyloxycarbonyl (Fmoc), for example.

Compounds of the present invention may contain one or more asymmetric centers and thus can occur as racemates and racemic mixtures, single enantiomers, diastereomeric mixtures and individual diastereomers. In certain embodiments, a single diastereomer is present. All possible stereoisomers of the compounds of the present invention are contemplated as being within the scope of the present invention. However, in certain aspects, particular diastereomers are contemplated. The chiral centers of the compounds of the present invention can have the S- or the R-configuration, as defined by the IUPAC 1974 Recommendations. In certain aspects, certain compounds of the present invention may comprise S- or R-configurations at particular carbon centers.

Synthetic techniques that may be used to prepare certain compounds of the present invention are provided in the Examples section. These techniques may be expanded to produce other aminoglycoside-lipid conjugates using techniques known in the art. Other synthetic techniques to prepare compounds of the present invention, such as precursors, as well as derivatives are well-known to those of skill in the art. For example, Smith and March, 2001 discuss a wide variety of synthetic transformations, reaction conditions, and possible pitfalls relating thereto, including amidation and esterification reactions. Methods of oxidizing a primary hydroxy position of an aminoglycoside such that is may be further reacted to produce a aminoglycoside-lipid conjugate are discussed in, for example, Kudyba et al., 2007, which is incorporated herein by reference in its entirety. Methods discussed therein may be adapted to prepare compounds of the present invention from commerically available starting materials.

As used herein, the term “patient” or “subject” refers to a living mammalian organism, such as a human, monkey, cow, sheep, goat, dogs, cat, mouse, rat, guinea pig, or transgenic species thereof. In certain embodiments, the patient or subject is a primate. Non-limiting examples of human subjects are adults, juveniles, infants and fetuses.

As used herein, a “multidrug resistant (MDR) bacteria” is resistant to two or more antimicrobial classes.

The claimed invention is also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like.

The use of the term “or” in the claims is used to mean “ and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Descriptions of well-known processing techniques, components, and equipment are omitted so as not to unnecessarily obscure the present methods and devices in unnecessary detail. Other objects, features and advantages of the present invention will become apparent from the following detailed description. It should be understood, however, that the detailed description and the specific examples, while indicating specific embodiments of the invention, are given by way of illustration only, as various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Structures of synthetic cationic lipids 1-16. Compounds 1-7 are neomycin B-derived cationic lipids containing an amine-based polycationic headgroup while neomycin B lipids 8-14 are comprised of a guanidinylated polycationic headgroup. Compounds 15 and 16 are kanamycin A-derived cationic lipids bearing a C₁₆-tail. TFA=trifluoroacetate.

### DESCRIPTION OF THE ILLUSTRATIVE EMBODIMENTS

Recently, it has been shown that neomycin B-derived cationic lipids prepared by conjugation of C₁₆- or C₂₀-lipid tails to the cationic head group of neomycin B restored antibacterial activity against methicillin-resistant S. aureus (MRSA) (MIC=8 mg/L) when compared to neomycin B (MIC=256 mg/L) (Bera et al., 2008). Here, the antibacterial activities of a novel class of cationic lipids in which guanidinylated neomycin B and kanamycin A as well as unmodified kanamycin A form the headgroup of a cationic lipid are shown. Neomycin B and kanamycin A differ in the number of cationic charges and size. Moreover, conversion of the amino groups into a guanidino function increases the basicity of the cationic headgroups.

Conjugation of neomycin B- and kanamycin A-derived polyamine and polyguanidinylated headgroups to hydrophobic lipid tails restore MRSA activity in both aminoglycosides and MRSE activity in kanamycin A. Optimal Gram-positive activity is achieved by conjugation of the polyamine-based headgroup to saturated C₁₆- or C₂₀-lipid tails. In the case of neomycin B-derived polyguanidinylated headgroups, induction of Gram-positive activity can be achieved by conjugation to shorter C₁₂-lipid tails. Moreover, guanidinylation of the polycationic headgroup in neomycin B-derived cationic lipids enhances antibacterial activity against neomycin B-, kanamycin A- and gentamicin-resistant P. aeruginosa strains as well as MRSA. Furthermore, polyguanidinylated aminoglycoside-based lipids display reduced hemolytic activity when compared to their polyamine analogs.

### A. AMINOGLYCOSIDE-LIPID CONJUGATES

An “aminoglycoside-lipid conjugate” is a cationic lipid in which a multiple charged cationic head group (the aminoglycoside) is linked to a lipid moiety, wherein the hydrophobicity of the aminoglycoside-lipid conjugate is greater than the hydrophobicity of the aminoglycoside in the absence of the lipid moiety. In certain embodiments, a aminoglycoside-lipid conjugate may comprise an aminoglycoside moiety having 3, 4, 5, or 6 or more positive charges, or any range derivable therein. Methods of measuring hydrophobicity are described herein. Non-limiting examples of aminoglycoside-lipid conjugates are compounds 8, 9, 10, 11, 12, 13, 14, 15, and 16, as shown herein.

1. Aminoglycosides

As used herein, an “aminoglycoside” refers a large and diverse class of antibiotics that characteristically contain two or more aminosugars linked by glycosidic bonds to an aminocyclitol component. Examples of aminoglycosides are neomycin, kanamycin, tobramycin, neamine, streptomycin, sisomycin and others. An “aminoglycoside antibiotic” or “AA” refers to a class of aminoglycosides that exhibit concentration-dependent antibacterial activity. See, e.g., Hooper, 1982; Haddad et al., 2001.

In any embodiment of the present invention, an aminoglycoside may be further defined as an aminoglycoside antibiotic. Aminoglycoside antibiotics are well-known in the art, and carry up to six amino groups which are predominantly charged at physiological pH (Sitaram and Nagaraj, 2002; Gordon et al., 1994). In certain embodiments, an aminoglycoside antibiotic of the present invention is further defined as a neomycin, a kanamycin, paromomycin, amikacin, a gentamicin, netilmycin, a streptomycin, tobramycin, sisomycin, a hygromycin, or a spectinomycin. In certain embodiments, the aminoglycoside antibiotic comprises a primary hydroxy position, such as in a neomycin, a kanamycin, amikacin, a streptomycin, tobramycin or a hygromycin. In particular embodiments, the aminoglycoside antibiotic is further defined as a neomycin or a kanamycin.

2. Lipids

In some embodiments, “lipid” or “lipid moiety” (used interchangeably) refers to a straight-chain hydrocarbon radical having 5 carbons or higher, wherein the radical may comprise single, double, and/or triple bonds. In other embodiments, the “lipid” or “lipid moiety” may be cyclic or aromatic. In certain embodiments, the straight-chain hydrocarbon radical has between 5 and 45 carbon atoms. In certain embodiments, a lipid may comprise only single bonds. In certain embodiments, a lipid may comprise 20 or fewer double bonds. In certain embodiments, a lipid may comprise at most or at least 19, 18, 17, 16, 15, 14, 13, 12, 11, 10, 9, 8, 7, 6, 5, 4, 3, 2, or 1 double bond(s), or any range derivable therein. In certain embodiments, a lipid may comprise 10 or fewer triple bonds. In certain embodiments, a lipid may comprise at most or at least 9, 8, 7, 6, 5, 4, 3, 2, or 1 triple bond(s), or any range derivable therein. In certain embodiments, a lipid may be of the formula CₐH₂ₐ₊₁, wherein a is 5-45. In certain embodiments, a is at least or at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or higher, or any range derivable therein. In certain embodiments, a lipid may be of the formula CₐH₂ₐ₋ₓ, wherein a is as defined above and x is an odd number such that 2a−x≧0. In certain embodiments, a lipid may be of the formula CₐH₂ₐ₋₁, wherein a is as described above. In certain embodiments, a lipid may be of the formula CₐH₂ₐ₋₃, wherein a is as described above. Non-limiting examples of lipids include —C₅H₁₁, —C₁₁H₂₃, —C₁₅H₃₁, —C₁₉H₃₉ and —C₁₇H₃₁. In certain embodiments, the lipid comprises pyrene or cholic acid. An aminoglycoside-lipid conjugate may comprise more than one lipid.

### B. BACTERIAL INFECTIONS

In certain aspects of the invention, the compositions are useful for the treatment and/or prevention of bacterial infections. The bacterial infection may be caused by a gram positive or gram negative bacterium. In some embodiments, the bacteria causing the bacterial infection is a multi-drug resistant bacteria.

The term “gram-negative bacteria” or “gram-negative bacterium” as used herein is defined as bacteria which have been classified by the Gram stain as having a red stain. Gram-negative bacteria have thin walled cell membranes consisting of a single layer of peptidoglycan and an outer layer of lipopolysacchacide, lipoprotein, and phospholipid. Exemplary organisms include, but are not limited to, Enterobacteriacea consisting of Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary Gram-negative organisms not in the family Enterobacteriacea include, but are not limited to, Pseudomonas aeruginosa, Stenotrophomonas maltophilia, Burkholderia, Cepacia, Gardenerella, Vaginalis, and Acinetobacter species.

The term “gram-positive bacteria” or “gram-positive bacterium” as used herein refers to bacteria, which have been classified using the Gram stain as having a blue stain. Gram-positive bacteria have a thick cell membrane consisting of multiple layers of peptidoglycan and an outside layer of teichoic acid. Exemplary organisms include, but are not limited to, Staphylococcus aureus, coagulase-negative staphylococci, streptococci, enterococci, corynebacteria, and Bacillus species.

### C. PHARMACEUTICAL COMPOSITIONS

In some aspects, the present invention provides a pharmaceutical composition comprising an aminoglycoside-lipid conjugate and a pharmaceutically acceptable carrier. The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, therapeutic compositions is contemplated. Supplementary active ingredients, such as other anti-microbial agents, can also be incorporated into the compositions.

The pharmaceutical compositions comprising the aminoglycoside-lipid conjugate may comprise different types of carriers depending on whether it is to be administered in solid, liquid or aerosol form, and whether it need to be sterile for such routes of administration as injection. The compositions of the present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (See, for example, Remington's Pharmaceutical Sciences, 18th Ed. Mack Printing Company, 1990, incorporated herein by reference).

The pharmaceutical compositions comprising the aminoglycoside-lipid conjugate may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for topical administrations or formulated for alimentary administrations such as drug release capsules and the like.

Further in accordance with some aspects of the present invention, the composition suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art. In one embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the active compound may comprise between about 2, 3, 4, 5, 10, 15, 20, 25, 30, or 40% to about 75, 74, 73, 72, 71, 70, 65, 60, 55, or 50% of the weight of the unit, and any range derivable therein. In other embodiments, the active compound may comprise between about 25% to about 60% of the weight of the unit, for example. Naturally, the amount of active compound(s) in each therapeutically useful composition may be prepared is such a way that a suitable dosage will be obtained in any given unit dose of the compound. Factors such as solubility; bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight; about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### D. THERAPEUTIC APPLICATIONS

In certain aspects of the invention, the compositions are useful for the treatment and/or prevention of bacterial infections. In some embodiments, the present invention provides a method of treating and/or preventing a bacterial infection comprising administering to the subject an effective amount of an aminoglycoside-lipid conjugate.

The term “effective concentration” or “effective amount” means that a sufficient amount of the antimicrobial agent is added to decrease, prevent or inhibit the growth of bacterial organisms or bacterial colonization. The amount will vary for each compound and upon known factors such as pharmaceutical characteristics; the type of medical device; age, sex, health and weight of the recipient; and the use and length of use. It is within the skilled artisan's ability to relatively easily determine an effective concentration for each compound. In some embodiments, the aminoglycoside-lipid conjugate may be employed in a method of the present invention such that the effective amount of the aminoglycoside-lipid conjugate is less than the effective amount of the corresponding aminoglycoside. In some embodiments, the effective amount is between 0.1 and 256 μg/mL, or any range derivable in between. In particular embodiments, the effective amount is ≦64 μg/mL. In other embodiments, the effective amount is 1, 2, 4, 8, 16, 32, 64, 128, or 256 μg/mL.

The terms “inhibiting” or “reducing” or any variation of these terms, when used in the claims and/or the specification, includes any measurable decrease or complete inhibition to achieve a desired result. For example, there may be a decrease of 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of bacterial infection following administration of a aminoglycoside-lipid conjugate of the present invention. In a further example, following administering of a aminoglycoside-lipid of the present invention, a patient suffering from a bacterial infection may experience a reduction the number and/or intensity of symptoms of the infection. Non-limiting examples of typical symptoms associated with a bacterial infection include elevated temperature, sweating, chills, and/or excess white blood cells compared to a normal range.

“Treatment” or “treating” includes (1) inhibiting a disease, condition, or infection in a subject or patient experiencing or displaying the pathology or symptomatology of the disease, condition, or infection (e.g., arresting further development of the pathology and/or symptomatology), (2) ameliorating a disease, condition, or infection in a subject or patient that is experiencing or displaying the pathology or symptomatology of the disease, condition, or infection (e.g., reversing the pathology and/or symptomatology), and/or (3) effecting any measurable decrease in a disease, condition, or infection in a subject or patient that is experiencing or displaying the pathology or symptomatology of the disease, condition, or infection. Aminoglycoside-lipid conjugates may be employed for treatment purposes in any embodiment herein, such as treatment of a bacterial infection.

“Prevention” or “preventing” includes: (1) inhibiting the onset of a disease, condition, or infection in a subject or patient which may be at risk and/or predisposed to the disease, condition, or infection but does not yet experience or display any or all of the pathology or symptomatology of the disease, condition, or infection, and/or (2) slowing the onset of the pathology or symptomatology of a disease, condition, or infection in a subject of patient which may be at risk and/or predisposed to the disease, condition, or infection but does not yet experience or display any or all of the pathology or symptomatology of the disease, condition, or infection.

In further embodiments, the method further comprises diagnosing the subject as needing treatment for the bacterial infection prior to administering the composition. Such diagnostic methods are well known to those having skill in the art.

1. Treatment Regimens

Treatment regimens may vary as well, and depend on the stage of bacterial infection and its consequences. The clinician will be best suited to make decisions on the best regimen to use based on the positive determination of the existing bacterial infection, the use of antibiotics and the known efficacy and toxicity (if any) of the therapeutic formulations.

The improvement is any observable or measurable improvement. Thus, one of skill in the art realizes that a treatment may improve the patient or subject's condition, but may not be a complete cure of the disease.

The composition of the present invention is utilized to markedly inhibit, reduce, prevent, abrogate, or minimize bacterial colonization, bacterial translocaton and/or bacterial invasion into host tissues. Reduction, abrogation, minimization or prevention of microbial growth is achieved by using an effective concentration such that the concentration is effective to reduce the growth or colonization or translocation into host tissues or invasion into host of the microbes by at least 10%, 20%, 30%, 40%, 50%, 60%, 70%, 80%, 90%, 100% or any range therebetween.

2. Combination Treatments

In order to increase the effectiveness of the composition, it may be desirable to combine these compositions and methods of the invention with a known agent effective in the treatment or prevention of bacterial infections, e.g., antibiotics. Antibacterial agents and classes thereof that may be co-administered .with a compound of the present invention include, without limitation, penicillins and related drugs, carbapenems, cephalosporins and related drugs, aminoglycosides, bacitracin, gramicidin, mupirocin, chloramphenicol, thiamphenicol, fusidate sodium, lincomycin, clindamycin, macrolides, novobiocin, polymyxins, rifamycins, spectinomycin, tetracyclines, vancomycin, teicoplanin, streptogramins, anti-folate agents including sulfonamides, trimethoprim and its combinations and pyrimethamine, synthetic antibacterials including nitrofurans, methenamine mandelate and methenamine hippurate, nitroimidazoles, quinolones, fluoroquinolones, isoniazid, ethambutol, pyrazinamide, para-aminosalicylic acid (PAS), cycloserine, capreomycin, ethionamide, prothionamide, thiacetazone, viomycin, eveminomicin, glycopeptide, glycylcycline, ketolides, oxazolidinone; imipenen, amikacin, netilmicin, fosfomycin, gentamicin, ceftriaxone, Ziracin, LY 333328, CL 331002, Linezolid, Synercid, Aztreonam, Metronidazole, Epiroprim, OCA-983, GV-143253, Sanfetrinem sodium, CS-834, Biapenem, A-99058.1, A-165600, A-179796, KA 159, Dynemicin A, DX8739, DU 6681; Cefluprenam, ER 35786, Cefoselis, Sanfetrinem celexetil, HGP-31, Cefpirome, HMR-3647, RU-59863, Mersacidin, KP 736, Rifalazil; Kosan, AM 1732, MEN 10700, Lenapenem, BO 2502A, NE-1530, PR 39, (L-arginyl-L-ariginyl-L-arginyl-L-prolyl-L-arginyl-L-prolyl-L-prolyl-L-tryosyl-L-leucyl-L-prolyl-L-arginyl-L-prolyl-L-arginyl-L-prolyl-L-prolyl-L-prolyl-L-phenylalanyl-L-phenylalanyl-L-prolyl-L-prolyl-L-arginyl-L-leucyl-L-prolyl-L-prolyl-L-arginyl-L-isoleucyl-L-prolyl-L-prolylglycyl-L-phenylalanyl-L-prolyl-L-prolyl-L-arginyl-L-phenyalanyl-L-prolyl-L-prolyl-L-arginyl-L-phenylalanyl-L-prolinamide), K130, OPC 20000, OPC 2045, Veneprim, PD 138312, PD 140248, CP 111905, Sulopenem, ritipenam acoxyl, RO-65-5788, Cyclothialidine, Sch-40832, SEP-132613, micacocidin A, SB-275833, SR-15402, SUN A0026, TOC 39, carumonam, Cefozopran, Cefetamct pivoxil, and T 3811.

In some embodiments, the composition of the present invention may precede, be co-current with and/or follow the other agent(s) by intervals ranging from minutes to weeks. In embodiments where the composition of the present invention, and other agent(s) are applied separately to a cell, tissue or organism, one would generally ensure that a significant period of time did not expire between the time of each delivery, such that the composition and agent(s) would still be able to exert an advantageously combined effect on the cell, tissue or organism.

Various combination regimens of the composition and one or more agents are employed. One of skill in the art is aware that the composition of the present invention and agents can be administered in any order or combination. In other aspects, one or more agents may be administered substantially simultaneously, or within about minutes to hours to days to weeks and any range derivable therein, prior to and/or after administering the composition.

Administration of the composition to a cell, tissue or organism may follow general protocols for the administration of antimicrobial therapeutics, taking into account the toxicity, if any. It is expected that the treatment cycles would be repeated as necessary. In particular embodiments, it is contemplated that various additional agents may be applied in any combination with the present invention.

Pharmacological therapeutic agents and methods of administration, dosages, etc. are well known to those of skill in the art (See for example, the “Physicians Desk Reference,” Goodman & Gilman's “The Pharmacological Basis of Therapeuticsm” “Remington's Pharmaceutical Sciences,” and “The Merck Index, Eleventh Edition” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

### E. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis of Various Aminoglycoside-Lipid Conjugates

The guanidinylated aminoglycoside-derived cationic lipids 8-14 were prepared from the known aminoglycoside lipid conjugates 1-7 by guanidinylation of the amino groups using N,N′-diBoc-N′-triflylguanidine as previously described followed by deprotection with trifluoroacetic acid and purification on reversed-phase C18 silica (Bera et al., 2008; Baker et al., 2000). The same synthetic strategy was used to prepare kanamycin A-based polycationic lipids 15 and 16. The identity of the synthetic cationic lipids was assessed by electrospray ionization-mass spectrometry, ¹H nuclear magnetic resonance and ¹³C nuclear magnetic resonance and the purity (>95%) was assessed by elemental analysis (see Example 2).

NMR spectra were recorded on a Brucker Avance 300 spectrometer (300 MHz for ¹H NMR, 75 MHz for ¹³C) and AMX 500 spectrometer (500 MHz for ¹H NMR). Optical rotation was measured at a concentration of g/100 mL, with a Perkin-Elmer polarimeter (accuracy (0.002°). GC-MS analyses were performed on a Perkin-Elmer Turbomass-Autosystem XL. Analytical thin-layer chromatography was performed on precoated silica gel plates. Visualization was performed by ultraviolet light and/or by staining with ninhydrine solution in ethanol. Chromatographic separations were performed on a silica gel column by flash chromatography (Kiesel gel 40, 0.040-0.063 mm; Merck). Yields are given after purification, unless differently stated. When reactions were performed under anhydrous conditions, the mixtures were maintained under nitrogen. Compounds were named following IUPAC rules as applied by Beilstein-Institute AutoNom (version 2.1) software for systematic names in organic chemistry.

### General Procedure A for the Guanidinylation of Aminoglycosides

N,N′-DiBoc-N″-triflylguanidine (1) was purchased from Fluka. To a solution of aminoglycoside (5 amines, 0.054 mmol) in H₂O (0.5 mL) was added 1,4-dioxane (2.5 mL) and N,N′-diBoc-N″-triflylguanidine (1, 0.82 mmol) in alternating portions so the solution remained relatively clear. After 5 min, NEt₃ (0.82 mmol) was added at room temperature. After 3-4 days, the 1,4-dioxane was removed under reduced pressure. The remaining residue and H₂O was extracted with CH₂Cl₂ (3×10 mL), washed with H₂O and brine, and dried (MgSO₄). The fully guanidinylated product can be isolated by flash column chromatography (fcc) on silica gel (CH₂Cl₂/MeOH).

### General Procedure B for the Deprotection of Guanidinoglycosides

A solution of TFA/CH₂Cl₂ (1:1, 1 mL) was added to the protected guanidinoglycoside (0.041 mmol) at room temperature. After approximately 4 h, the solution was diluted with toluene, concentrated in vacuo, and dissolved in H₂O. Subsequent lyopholyzation of H₂O provided the deprotected guanidinoglycoside as a fluffy white powder.

### Procedure for the Coupling Reaction

To a solution of kanamycin or neomycin amine (1 eq.) in dry DMF, TBTU (2 eq.), lipidic acid (1 eq.) and DIPEA (3 equiv) were added and stirred at room temperature for 2 h. The reaction mixture was triturated with water and ethylacetate. The ethylacetate layer was washed with water, brine, dried over sodium sulfate and concentrated. The crude residue was purified using flash silica gel by eluting with MeOH/CH₂Cl₂. The spectroscopic data were given below.

### Synthesis of Kanamycin-A Lipid Conjugates

### Procedure for the Coupling Reaction

To a solution of Kanamycin A amine (1 eq.) in dry DMF, TBTU (2 equiv), hexadecanoic acid (1 eq) and DIPEA (3 equiv) were added and stirred at room temperature for 2 h. The reaction mixture was triturated with water and ethylacetate. The ethylacetate layer was washed with water, brine, dried over sodium sulfate and concentrated. The crude residue was purified using flash silica gel by eluting with MeOH/CH₂Cl₂. The spectroscopic data were given below.

### General Procedure for Final Deprotection

All of the BOC-protected aminoglycoside-based compounds were treated with 95% TFA for 3 min at 0° C. TFA was removed at reduced pressure. To the residue 2% methanol in ether was added and the solvent was decanted to get the solid kanamycin-lipid conjugates as salt. The spectroscopic data were given below.

### Example 2

### Characterization Data for Compounds 8-16 1,3,2′,6′,2′″,6′″-Hexaammonium-guanidinyl-5″-deoxy-5″-N-(hexanoyl)-neomycin hexakis(trifluoroacetate) (8)

Yield=91%; R_{f} 0.10 (NH₄OH/MeOH/CH₂Cl₂ 2:6:4); ¹H NMR (300 MHz, CD₃OD): δ 5.91 (d, 1H, J=4.2 Hz), 5.12 (d, 1H, J=4.2 Hz), 5.02 (s, 1H), 4.23 (t, 1H, J=6.1 Hz), 4.10 (t, 1H, J=5.1 Hz), 4.06 (d, 1H, J=3.2 Hz), 4.01 (t, 1H, J=3.2 Hz), 3.96 (dd, 1H, J=5.2, 9.0 Hz), 3.80 (m, 4H), 3.64 (m, 6H), 3.55 (m, 2H), 3.50 (m, 3H), 3.39 (m, 2H), 2.26 (t, 2H, J=7.1 Hz), 2.15 (dt, 1H, J=2.9, 13.3 Hz), 1.76 (t, 1H, J=10.7 Hz), 1.61 (quintet, 2H, J=7.4 Hz), 1.34 (m, 4H), 0.93 (t, 3H, J=7.7 Hz); ¹³C NMR (75 MHz, CD₃OD): δ 177.1 (amide C═O), 159.6, 159.5, 159.3, 159.2, 158.6 (Guanidine C═NH), 111.5, 100.5, 97.1 (anomeric carbons), 87.8, 82.4, 82.4, 79.8, 77.5, 76.6, 75.7, 74.1, 72.0, 71.4, 70.9, 69.0, 57.1, 55.3, 53.6, 51.7, 43.1, 43.0, 42.0, 37.1, 33.8-23.4 (aliphatic CH₂), 14.5 (aliphatic CH₃); [α]_{D}²⁵=+22.0 (c 0.86, MeOH); ELMS: calcd for C₃₅H₇₀N₁₉O₁₃⁺ 964.53 Found: 964.53 [M+H]⁺; Anal. Calcd. for C₄₇H₇₅F₁₈N₁₉O₂₅ C, 34.25; H, 4.59; F, 20.75; N, 16.15; Found: C, 34.66; H, 4.81; F, 21.05; N, 16.43.

### 1,3,2′,6′,2′″,6′″-Hexaammonium-guanidinyl-5″-deoxy-5″-N-(dodecanoyl)-neomycin hexakis(trifluoroacetate) (9)

Yield=90%; R_{f} 0.10 (NH₄OH/MeOH/CH₂Cl₂ 2:6:4); ¹H NMR (300 MHz, CD₃OD): δ 5.92 (d, 1H, J=3.1 Hz), 5.10 (d, 1H, J=3.8 Hz), 5.02 (s, 1H), 4.21 (t, 1H, J=5.3 Hz), 4.09 (t, 1H, J=4.1 Hz), 4.07 (m, 1H), 4.00 (t, 1H, J=3.1 Hz), 3.94 (m, 1H), 3.80 (m, 4H), 3.64 (m, 4H), 3.55 (m, 2H), 3.48 (m, 4H), 3.39 (m, 1H), 3.34 (m, 2H), 2.25 (t, 2H, J=6.4 Hz), 2.13 (dd, 1H, J=4.7, 12.5 Hz), 1.72 (t, 1H, J=14.6 Hz), 1.60 (m, 2H), 1.29 (br s, 16H), 0.93 (t, 3H, J=5.8 Hz); ¹³C NMR (75 MHz, CD₃OD): δ 177.3 (amide C═O), 159.6, 159.5-159.2 (guanidine C═NH), 158.6, 111.5, 100.5, 97.1 (anomeric carbons), 87.8, 82.4, 82.4, 79.8, 77.5, 76.6, 75.7, 74.2, 74.1, 72.0, 71.4, 70.9, 69.0, 57.1, 55.3, 53.6, 51.7, 43.4, 43.0, 42.0, 37.2, 33.8-23.6 (aliphatic CH₂), 14.4 (aliphatic CH₃); [α]_{D}²⁵=+22.0 (c 1.75, MeOH); EIMS: calcd for C₄₁H₈₂N₁₉O₁₃⁺ 1048.63 Found: 1048.63 [M+H]⁺; Anal. Calcd. for C₅₃H₈₇F₁₈N₁₉O₂₅ C, 36.75; H, 5.06; F, 19.74; N, 15.36; Found: C, 36.87; H, 4.99; F, 19.91; N, 15.55.

### 1,3,2′,6′,2′″,6′″-Hexaammonium-guanidinyl-5″-deoxy-5″-N-(hexadecanoyl)-neomycin hexakis(trifluoroacetate) (10). Yield=93%; R_{f} 0.10 (NH₄OH/MeOH/CH₂Cl₂ 2:6:4)

¹H NMR (300 MHz, CD₃OD): δ 5.91 (d, 1H, J=2.4 Hz), 5.60 (m, 1H), 5.12 (t, 1H, J=3.8 Hz), 5.02 (m, 1H), 4.23 (t, 1H, J=6.3 Hz), 4.09 (m, 2H), 4.01 (t, 1H, J=2.7 Hz), 3.95 (m, 1H), 3.80 (m, 4H), 3.63 (m, 5H), 3.55 (m, 2H), 3.50 (m, 3H), 3.37 (m, 2H), 2.25 (t, 2H, J=7.8 Hz), 2.11 (dt, 1H, J=3.3, 13.3 Hz), 1.75 (m, 1H), 1.60 (m, 2H), 1.29 (br s, 24H), 0.93 (t, 3H, J=6.1 Hz); ¹³C NMR (75 MHz, CD₃OD): δ 177.3 (amide C═O), 159.6, 159.5-159.2, 158.6 (guanidine C═NH), 111.5, 100.0, 97.1 (anomeric carbons), 87.8, 82.4, 79.8, 77.5, 76.6, 75.7, 74.1, 72.0, 71.4, 70.9, 68.9, 57.2, 55.3, 53.6, 51.7, 43.1, 43.0, 42.0, 37.2, 33.0-23.6 (aliphatic CH₂), 14.4 (aliphatic CH₃); [α]_{D}²⁵=+57.0 (c 0.8, MeOH); EIMS: calcd for C₄₅H₉₀N₁₉O₁₃⁺ 1104.69 Found: 1104.69 [M+H]⁺; Anal. Calcd. for C₅₇H₉₅F₁₈N₁₉O₂₅ C, 38.28; H, 5.35; F, 19.12; N, 14.88; Found: C, 38.44; H, 5.67; F, 19.31; N, 15.11.

### 1,3,2′,6′,2′″,6′″-Hexaammonium-guanidinyl-5″-deoxy-5″-N-(nonadecanoyl)-neomycin hexakis(trifluoroacetate) (11)

Yield=93%; R_{f} 0.10 (NH₄OH/MeOH/CH₂Cl₂ 2:6:4); ¹H NMR (300 MHz, CD₃OD): δ 5.91 (d, 1H, J=2.4 Hz), 5.10 (d, 1H, J=4.0 Hz), 5.07 (s, 1H), 4.21 (t, 1H, J=5.2 Hz), 4.09 (m, 2H), 4.01 (t, 1H, J=3.3 Hz), 3.95 (m, 1H), 3.76 (m, 4H), 3.62 (m, 6H), 3.54 (m, 1H), 3.48 (m, 3H), 3.37 (m, 2H), 3.15 (t, 1H, J=6.4 Hz), 2.25 (t, 2H, J=6.8 Hz), 2.10 (d, 1H, J=10.4 Hz), 1.80 (m, 1H), 1.59 (d, 2H, J=6.8 Hz), 1.28 (m, 32H), 0.90 (t, 3H, J=5.0 Hz); ¹³C NMR (75 MHz, CD₃OD): δ 177.3 (amide C═O), 159.6, 159.5-159.2, 158.6 (guanidine C═NH), 111.5, 100.0, 97.1 (anomeric carbons), 87.8, 82.4, 79.8, 77.4, 76.7, 75.0, 74.3, 74.1, 73.0, 72.0, 71.4, 70.9, 68.9, 64.3, 57.1, 55.3, 53.6, 43.4, 42.0, 37.2-23.6 (aliphatic CH₂), 14.4 (aliphatic CH₃); [α]_{D}²⁵=+36.0 (c 0.45, MeOH); EIMS: calcd for C₄₉H₉₈N₁₉O₁₃⁺ 1160.75 Found: 1160.75 [M+H]⁺; Anal. Calcd. for C₆₁H₁₀₃F₁₈N₁₉O₂₅ C, 39.72; H, 5.63; F, 18.54; N, 14.43; Found: C, 39.88; H, 5.90; F, 18.63; N, 14.61.

### 1,3,2′,6′,2′″,6′″-Hexaammonium-guanidinyl-5″-deoxy-5″-N-(9Z,12Z,octadeca-di-ene-oyl)-neomycin hexakis(trifluoroacetate) (12)

Yield=93%; R_{f} 0.10 (NH₄OH/MeOH/CH₂Cl₂ 2:6:4); ¹H NMR (300 MHz, CD₃OD): δ 5.91 (d, 1H, J=2.3 Hz), 5.10 (d, 2H, J=5.4 Hz), 5.02 (s, 2H), 4.93 (m, 2H), 4.21 (t, 1H, J=5.0 Hz), 4.09 (m, 2H), 4.01 (t, 1H, J=3.1 Hz), 3.95 (dd, 1H, J=4.7, 10.1 Hz), 3.79 (m, 2H), 3.76 (m, 2H), 3.63 (m, 5H), 3.50 (m, 5H), 3.39 (m, 1H), 3.20 (m, 1H), 3.15 (t, 1H, J=9.7 Hz), 2.25 (t, 2H, J=7.8 Hz), 2.11 (d, 1H, J=12.5 Hz), 2.01 (m, 1H), 1.71 (m, 2H), 1.60 (m, 4H), 1.33 (m, 16H), 0.93 (t, 3H, J=6.4 Hz); ¹³C NMR (75 MHz, CD₃OD): δ 177.1 (amide C═O), 159.6, 159.5-159.2, 158.6 (guanidine C═NH), 111.5, 100.0, 97.1 (anomeric carbons), 87.8, 82.4, 79.8, 77.4, 76.9, 75.8, 74.2, 74.1, 72.0, 71.9, 71.4, 70.9, 68.9, 57.2, 55.3, 53.6, 51.7, 43.6, 43.0, 37.2, 33.8-23.6 (aliphatic CH₂) 14.4 (aliphatic CH₃); [α]_{D}²⁵=+26.0 (c 0.65, MeOH); EIMS: calcd for C₄₇H₉₀N₁₉O₁₃⁺ 1127.69 Found: 1127.69 [M+H]⁺; Anal. Calcd. for C₅₉H₉₅F₁₈N₁₉O₂₅ C, 39.10; H, 5.28; F, 18.87; N, 14.68; Found: C, 39.33; H, 5.46; F, 19.21; N, 14.90.

### 1,3,2′,6′,2′″,6′″-Hexaammonium-guanidinyl-5″-deoxy-5″-N-((4-pyrenyl)-butanoyl)-neomycin hexakis(trifluoroacetate) (13)

Yield=89%; R_{f} 0.12 (NH₄OH/MeOH/CH₂Cl₂ 2:6:4); ¹H NMR (300 MHz, CD₃OD): δ 8.31 (d, 1H, J=9.8 Hz), 8.17 (m, 4H), 8.03 (m, 1H), 8.04 (m, 2H), 7.90 (d, 1H, J=6.2 Hz), 5.90 (d, 1H, J=6.2 Hz), 5.11 (d, 1H, J=5.9 Hz), 5.03 (s, 1H), 4.27 (t, 1H, J=5.7 Hz), 4.13 (t, 1H, J=4.2 Hz), 4.06 (m, 1H), 4.02 (t, 1H, J=2.2 Hz), 3.97 (m, 2H), 3.77 (m, 3H), 3.62 (m, 5H), 3.51 (m, 2H), 3.41 (m, 6H), 2.47 (t, 2H, J=6.8 Hz), 2.17 (m, 4H), 1.71 (m, 2H); ¹³C NMR (75 MHz, CD₃OD): δ 176.9 (amide C═O), 159.6-158.5 (guanidine C═NH), 137.3-124.3 (aromatic carbons), 124.1-112.4 (q with J¹_{CF}˜292.0 Hz), 111.3, 100.1, 97.1 (anomeric carbons), 87.8, 82.4, 79.9, 77.4, 76.7, 75.7, 74.2, 74.0, 72.0, 71.5, 70.9, 68.8, 57.1, 55.1, 53.6, 51.7, 43.4, 43.1, 42.1, 36.8, 33.9, 33.7, 29.0; [α]_{D}²⁵=+20.0 (c 0.93, MeOH); EIMS: calcd for C₄₉H₇₄N₁₉O₁₃⁺ 1136.56 Found: 1136.56 [M+H]⁺; Anal. Calcd. for C₆₁H₇₉F₁₈N₁₉O₂₉ C, 40.25; H, 4.37; F, 18.79; N, 14.62; Found: C, 40.70; H, 4.29; F, 19.01; N, 14.87.

### 1,3,2′,6′,2′″,6′″-Hexaammonium-guanidinyl-5″-deoxy-5″-N-(cholanoyl)-neomycin hexakis(trifluoroacetate) (14)

Yield=90%; R_{f} 0.10 (NH₄OH/MeOH/CH₂Cl₂ 2:9:4); ¹H NMR (300 MHz, CD₃OD): δ 5.91 (d, 1H, J=2.5 Hz), 5.09 (d, 1H, J=4.2 Hz), 5.00 (s, 2H), 4.21 (quintet, 1H, J=4.5 Hz), 4.07 (m, 2H), 4.01 (t, 2H, J=3.9 Hz), 3.96 (dd, 1H, J=4.5, 8.8 Hz), 3.83 (m, 5H), 3.63 (m, 6H), 3.51 (m, 5H), 3.38 (m, 2H), 2.38-2.10 (m, 6H), 2.01 (t, 1H, J=5.1 Hz), 1.94 (m, 1H), 1.84 (m, 7H), 1.69 (m, 7H), 1.40 (m, 4H), 1.04-0.72 (m, 9H); ¹³C NMR (75 MHz, CD₃OD): δ 177.7 (amide C═O), 159.6-158.5 (guanidine C═NH), 164.0-162.7 (TFA, q with J²_{CF}˜34.8 Hz), 124.1-112.4 (q with J¹_{CF}˜292.0 Hz), 111.4, 100.1, 97.1 (anomeric carbons), 87.8, 79.7, 76.6, 75.6, 74.2, 74.1, 73.2, 72.8, 72.3, 72.0, 71.4, 70.8, 69.8, 69.3, 69.2, 66.4, 57.1, 55.2, 53.6, 51.7, 48.4, 47.8, 47.4, 43.1, 39.7, 39.4, 36.9, 36.4-24.2 (aliphatic CH₂), 18.1, 13.0 (CH₃); [α]_{D}²⁵=+29.0 (c 1.4, MeOH); EIMS: C₅₃H₉₈N₁₉O₁₆⁺ 1256.74 Found: 1256.74 [M+H]⁺; Anal. Calcd. for C₆₅H₁₀₃F₁₈N₁₉O₂₈ C, 40.23; H, 5.35; F, 17.62; N, 13.71; Found: C, 40.54; H, 5.31; F, 17.73; N, 13.89.

### 6″-(2,4,6-Triisopropylbenzenesulfonyl)-1,3,6′,3″-tetra-N-(tert-butoxycarbonyl)kanamycin A (17)

To a solution of 1,3,6′,3″-tetra-N-(tert-butoxycarbonyl)kanamycin A (200 mg, 0.59 mmol) in 8.8 mL of pyridine, 2,4,6-triisopropylbenzenesulfonyl chloride (1.2 g, 3.5 mmol, 6 equiv) was added in and the reaction mixture was stirred for two days. Solvent was removed at reduced pressure, the reaction mixture was partitioned between water and ethylacetate. The aqueous layer was extracted with EtOAc (3×50 mL) and the organic layers were combined and extracted with brine, dried over Na₂SO₄, and concentrated to a solid. Crude product was purified by silica gel chromatography (3% MeOH/DCM): yield 320 mg, 46%; Rf=0.69 (15% MeOH/DCM).

### 1,3,6′,3″-Tetra-N-(tert-butoxycarbonyl)-6″-azido-6″-deoxy-kanamycin A (18)

Compound 17 (150 mg, 0.130 mmol) was dissolved in 5 mL of DMF to which sodium azide (120 mg, 2.4 mmol, 20 equiv) was added, and the reaction mixture was heated at 60° C. overnight. After completion, the mixture was added to 150 mL of H₂O and extracted with EtOAc (3×150 mL). The organic layers were combined, washed with brine, dried over Na₂SO₄, and concentrated to a solid. The sample was purified by silica gel chromatography to get 18. Yields: 93%, R_{f}: 0.36 (MeOH/CH₂Cl₂ 1:12); IR (KBr disk) 2106.3 cm⁻¹ (N₃); ¹H NMR (300 MHz, CD₃OD): δ 5.11 (br s, 1H), 5.10 (br s, 1H), 4.33 (d, 1H, J=9.8), 3.74 (d, 2H, J=9.8 Hz), 3.64 (t, 1H, J=9.2 Hz), 3.69 (dd, 1H, J=4.3, 11.5 Hz), 3.59 (m, 2H), 3.56 (d, 1H, J=2.5 Hz), 3.51 (d, 1H, J=2.5 Hz), 3.45 (dd, 2H, J=4.3, 11.5 Hz), 3.41 (d, 2H, J=3.8 Hz), 3.38 (t, 3H, J=3.6 Hz), 3.21 (t, 1H, J=9.4 Hz), 2.09 (br d, 1H, J=12.2 Hz), 1.55 (m, 1H), 1.47 (2 s, 36H); ¹³C NMR (75 MHz, CD₃OD): δ 156.4, 155.6 (×2), 154.9, 101.4, 97.6 (anomeric carbons), 84.6, 79.6, 78.4 (×3), 78.0, 75.1, 72.7, 72.2, 71.2, 70.4, 70.1, 68.2, 55.5, 50.7, 49.9, 49.1, 40.8, 34.7, 27.9-27.6; EIMS: calcd for C₃₈H₆₇N₄NaO₁₈⁺ 932.44 Found: 932.38 [M+Na]⁺; [α]_{D}²⁵=+45.0 (c 0.97, MeOH); Anal. Calcd. for C₃₈H₆₇N₇O₁₈ C, 50.16; H, 7.42; N, 10.77; Found: C, 50.34; H, 7.42; N, 11.09.

### 1,3,6′,3″-Tetra-N-(tert-butoxycarbonyl)-6″-amino-6″-deoxy-kanamycin A (19)

The solution of kanamycin azide 18 (500 mg, 0.549 mmol) and 10% Pd(OH)₂/C in methanol (25 mL) was hydrogenated at normal temperature and pressure for 4 h and then filtered through celite. Filtrate was concentrated and the residue was purified on flush column chromatography by using MeOH/CH₂Cl₂ (1:9) to afford 19 (411 mg, 85%), as a white solid. ¹H NMR (300 MHz, CD₃OD-d₆): δ 5.06 (d, 1H, J=3.2 Hz), 5.03 (d, 1H, J=3.5 Hz), 4.52 (m, 1H), 4.34 (m, 1H), 4.23 (t, 1H, J=10.6 Hz), 4.03 (d, 1H, J=10.6 Hz), 3.80 (m, 1H), 3.72 (m, 1H), 3.69 (m, 1H), 3.66 (m, 1H), 3.62 (m, 2H), 3.52 (m, 2H), 3.46 (m, 1H), 3.42 (m, 2H), 3.22 (t, 1H, J=10.0 Hz), 3.00 (q, 1H, J=10.0 Hz), 2.10 (m, 1H), 1.52 (m, 1H), 1.46 (2 s, 36H); ¹³C NMR (75 MHz, CD₃OD): δ 159.2, 158.7, 157.6, 157.2, 102.5, 99.8 (anomeric carbons), 87.4, 85.9, 83.1 (×4), 80.4, 74.0, 73.4, 72.0, 71.2, 70.7, 69.7, 56.8, 51.6, 50.4, 41.4, 35.4, 28.9-28.8; EIMS: calcd. for C₃₈H₇₀N₅O₁₈⁺ 884.47 Found: 884.44 (M+H)⁺; [α]_{D}²⁵=+39.0 (c 0.87, MeOH); EIMS: C₃₈H₆₉N₅NaO₁₈⁺ 906.46 Found: 906.65 [M+Na]⁺; Anal. Calcd. for C₃₈H₆₉N₅O₁₈ C, 51.63; H, 7.87; N, 7.92; Found: C, 52.25; H, 8.14; N, 7.92.

### 6″-N-(Hexadecanoyl)-1,3,6′,3″-tetra-N-(tert-butoxycarbonyl)-6″-deoxy-kanamycin (20)

Yield=87%; R_{f} 0.31 (MeOH/CH₂Cl₂ 1:15); [α]_{D}²⁵=50.0 (c 0.7, MeOH); ¹H NMR (300 MHz, CD₃OD): δ 5.09 (d, 1H J=2.5 Hz), 5.04 (d, 1H J=3.5 Hz), 4.21 (t, 1H, J=8.2 Hz), 3.71 (dd, 1H, J=9.6, 16.6 Hz), 3.64 (d, 3H, J=9.4 Hz), 3.58 (t, 2H, J=7.7 Hz), 3.54-3.36 (m, 8H), 3.24 (d, 1H, J=9.3 Hz), 3.19 (t, 1H, J=9.0 Hz), 2.23 (t, 2H, J=7.7 Hz), 2.06 (br d, 1H, J=11.2 Hz), 1.62 (m, 3H), 1.47 (2 s, 36H), 1.26 (s, 24H), 0.90 (t, 3H, J=6.7 Hz); ¹³C NMR (75 MHz, CD₃OD): δ 176.7 (Amide C═O), 159.3, 159.1, 158.0, 157.7, 102.9, 100.3 (anomeric carbons), 86.2, 81.8, 80.6, 80.4, 80.1, 77.1, 74.6, 74.0, 72.4, 72.1, 72.0, 71.8, 71.3, 57.4, 52.3, 50.9, 42.1, 41.9, 37.3, 35.9-23.7 (aliphatic CH₂), 14.6 (CH₃); EIMS: calcd. for C₅₄H₉₉N₅NaO₁₉⁺ 1141.68 Found: 1141.57 (M+Na)⁺; [α]_{D}²⁵=+54.0 (c 1.23, MeOH); EIMS: C₅₄H₉₉N₅NaO₁₉⁺ 1144.69 Found: 1145.10 [M+Na]⁺; Anal. Calcd. for C₅₄H₉₉N₅O₁₉ C, 57.79; H, 8.89; N, 6.24; Found: C, 57.79; H, 8.89; N, 6.24.

### 1,3,6′,3″-Tetraammonium-6″-deoxy-6″-N-(hexadecanoyl)-kanamycin-tetrakis-(trifluoroacetate) (15)

Yield=90%; R_{f} 0.20 (NH₄OH/MeOH/CH₂Cl₂, 1:5:5); [α]_{D}²⁵=49.1 (c 0.1, MeOH); ¹H NMR (300 MHz, CD₃OD): δ 5.46 (d, 1H J=3.5 Hz), 5.04 (d, 1H J=3.5 Hz), 4.06 (dt, 1H, J=2.2, 9.4 Hz), 3.95 (m, 1H), 3.84 (m, 4H), 3.71 (t, 2H, J=9.3 Hz), 3.64 (dd, 2H, J=4.8, 14.4 Hz), 3.61 (d, 1H, J=6.9 Hz), 3.54 (dd, 2H, J=3.6, 9.3 Hz), 3.50 (m, 1H), 3.39 (dd, 3H, J=2.3, 9.3 Hz), 3.22 (t, 1H, J=9.7 Hz), 3.02 (dd, 1H, J=9.4, 13.2 Hz), 2.53 (dt, 1H, J=3.6, 12.3 Hz), 2.25 (t, 2H, J=7.7 Hz), 2.01 (m, 1H), 1.62 (quintet, 2H, J=6.9 Hz), 1.30 (s, 24H), 0.90 (t, 3H, J=6.7 Hz); ¹³C NMR (75 MHz, CD₃OD): δ 177.7 (Amide C═O), 164.0-162.7 (TFA carbons, q with J¹_{CF}˜34.8 Hz), 124.1-112.4 (TFA carbons, q with J¹_{CF}˜292.0 Hz), 102.5, 97.0 (anomeric carbons), 85.4, 81.9, 74.4, 73.5, 73.5, 73.0, 72.9, 70.6, 70.3, 68.4, 56.3, 51.8, 42.2, 40.8, 37.0, 33.1-23.8 (aliphatic CH₂) 14.6 (CH₃); [α]_{D}²⁵=+43.0 (c 0.69, MeOH); EIMS: calcd. for C₃₄H₆₈N₅O₁₁⁺ 722.49 Found: 722.09 (M+H)⁺; Anal. Calcd. for C₄₂H₇₁F₁₂N₅O₁₉ C, 42.82; H, 6.07; F, 19.35; N, 5.95; Found: C, 43.21; H, 6.11; F, 19.48; N, 6.07.

### 1,3,6′,3″-Tetraammonium-guanidinyl-6″-deoxy-6″-N-(hexadecanoyl)-kanamycin-tetrakis-(trifluoroacetate) (16)

Yield=90%; R_{f} 0.10 (NH₄OH/MeOH/CH₂Cl₂ 2:10:4); ¹H NMR (300 MHz, CD₃OD): δ 5.43 (br s, 1H), 5.08 (br s, 1H), 4.74 (m, 1H), 4.58 (d, 1H, J=11.7 Hz), 4.02 (m, 1H) 3.91-3.39 (m, 14H), 2.75 (m, 1H), 2.54 (t, 1H, J=11.2 Hz), 2.43 (t, 1H, J=6.3 Hz), 2.28 (dt, 1H, J=6.8, 12.2 Hz), 2.00 (m, 1H), 1.85 (m, 1H), 1.66 (m, 2H), 1.27 (s, 22H), 0.89 (t, 3H, J=8.7 Hz); ¹³C NMR (75 MHz, DMSO-d₆): δ 174.9 (Amide C═O), 159.8, 159.3, 158.7, 158.1 (guanidine C═NH), 99.3, 98.4 (anomeric carbons), 85.3, 82.0, 80.7, 74.2, 73.1, 72.6, 71.9, 70.3, 66.2, 64.1, 59.1, 56.0, 51.1, 45.0, 43.4, 37.0, 34.0-23.6 (aliphatic CH₂) 14.4 (CH₃); [α]_{D}²⁵=+52.0 (c 0.102, MeOH); EIMS: C₃₈H₇₆N₁₃O₁₁⁺ 890.57 Found: 891.01 [M+H]⁺; Anal. Calcd. for C₄₆H₇₉F₁₂N₁₃O₁₉ C, 41.04; H, 5.92; F, 16.94; N, 13.53; Found: C, 41.33; H, 6.13; F, 17.14; N, 14.03.

### Example 3

### Antibacterial Testing of Various Aminoglycoside-Lipid Conjugates

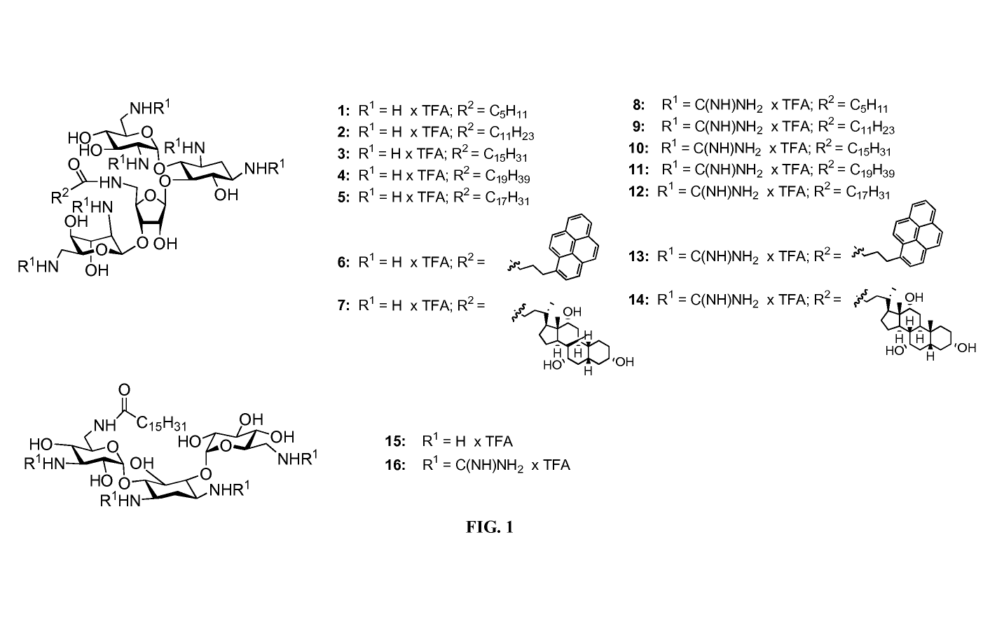

A total of 7 neomycin B-based and 2 kanamycin A-based polycationic lipids were synthesized (FIG. 1) and their antibacterial activities were assessed (Table 1). A variety of lipophilic moieties including C₆-, C₁₂-, C₁₆-, and C₂₀-, double-unsaturated C₁₈-, pyrene and cholic acid were selected to explore how the nature of the hydrophobic tails affects the antibacterial activity. Neomycin B and kanamycin A were selected due to their commercial availability in multigram quantities, low price and well documented resistance profiles. In addition, both aminoglycosides allow exploration of effects caused by structural differences in size and number of cationic charges. Gentamicin, neomycin B and kanamycin A served as positive controls.

American Type Culture Collection (ATCC) strains as well as clinical isolates from the Canadian Intensive Care Unit (CAN-ICU) study were used for susceptibility testing including including: S. aureus ATCC 29213, MRSA ATCC 33592, S. epidermidis ATCC 14990, methicillin-resistant Staphylococcus epidermidis (MRSE) (cefazolin-CZ MIC >32 mg/L) CAN-ICU 61589, E. faecalis ATCC 29212, E. faecium ATCC 27270, S. pneumoniae ATCC 49619, E. coli ATCC 25922, E. coli (gentamicin-resistant MIC >32 mg/L) CAN-ICU 61714, E. coli (amikacin MIC 32 mg/L) CAN-ICU 63074, P. aeruginosa ATCC 27853 and P. aeruginosa (gentamicin-resistant MIC >32 mg/L) CAN-ICU 62308 (Zhanel et al., 2008).

Antibacterial activity against Gram-positive and Gram-negative microorganisms was tested via broth macrodilution tests using CLSI methodology (Zhanel et al., 2008). Stock solutions of cationic lipids in water were brought to a concentration of 512 mg/L. Organisms were subcultured and isolated on blood agar, suspended in 3 mL of Mueller-Hinton broth at the turbidity of a 0.5M McFarland Standard, and diluted to approximately 10⁵ cfu/mL before introduction into tubes containing serially diluted lipopeptide antibiotic in Mueller-Hinton broth. Activity testing against S. pneumoniae used broth supplemented with laked horse blood to give 5% horse blood in experimental tubes. Turbidity of lipopeptide solution in broth required creation of control tubes lacking organisms serving as turbidity controls. All tubes were incubated overnight for 16-20 h at 37° C. Colony counts confirmed the tested inoculum at 10⁵ cfu/mL. All experiments were performed in duplicate. In-vitro toxicity was determined using a sheep red blood cell hemolytic assay as previously described (Dathe et al., 1996). Pilot experiments have shown that both human and sheep erythrocytes respond similarly in this hemolytic assay (data not shown).

### Determination of the MIC Values for Compounds 8-16

Bacterial isolates studied included both reference strains obtained from the American Type Culture Collection (ATCC) as well as clinical strains obtained from the CAN-ICU study which was a national surveillance study assessing the prevalence of antimicrobial resistant pathogens in Canadian intensive care units (Zhanel et al., 2008). Isolates were kept frozen in skim milk at −80° C. until minimum inhibitory concentration (MIC) testing was carried out. Following two subcultures from frozen stock, the in vitro activities of peptides were determined by macrobroth dilution in accordance with the Clinical and Laboratory Standards Institute (CLSI) 2006 guidelines (Zhanel et al., 2008). Stock solutions of peptides were prepared and dilutions made as described by CLSI. Test tubes contained doubling antimicrobial dilutions of cation adjusted Mueller-Hinton broth and inoculated to achieve a final concentration of approximately 5×10⁵ cfu/ml then incubated in ambient air for 24 hours prior to reading. Colony counts were performed periodically to confirm inocula. Quality control was regularly performed using ATCC QC organisms.

### Hemolytic Assay

In vitro toxicity was determined using a sheep red blood cell (erythrocytes) hemolytic assay (Dathe et al., 1996). Pilot experiments have shown that both human and sheep erythrocytes respond similarly in this hemolytic assay (data not shown). Erythrocytes were washed and resuspended in Tris buffered saline. The cell suspension was combined with varying concentrations (low to very high) of test and control antimicrobials (e.g., aminoglycosides and APTCs). The samples were centrifuged and the absorbance of the supernatants measured at 540 nm. Tris buffered saline and Triton X were used as negative and positive controls, respectively. The toxicity was assessed by percent hemolysis.

### Results

The antibacterial activities of a total of 7 neomycin B-based and 2 kanamycin A-based polycationic lipids were assessed (Table 1).

TABLE 1
MIC (mg/L) of neomycin B- and kanamycin A-based cationic lipids

| Control | | | | | | | | | | |
|---|---|---|---|---|---|---|---|---|---|---|
| Organism | Gentamicin | Neomycin B | 1 | 2 | 3 | 4 | 5 | 6 | 7 | 8 |
| S. aureus | 1 | 1 | 16 | 32 | 4 | 8 | 16 | 16 | 16 | 128 |
| ATCC29213 | | | | | | | | | | |
| MRSA | 2 | 256 | >512 | >256 | 8 | 8 | 32 | 256 | 128 | 256 |
| ATCC33592 | | | | | | | | | | |
| S. epidermidis | 0.25 | 0.25 | 2 | 4 | 2 | 4 | 4 | 2 | 8 | 64 |
| ATCC14990 | | | | | | | | | | |
| MRSE | 32 | 0.5 | 32 | 8 | 2 | 4 | 4 | 64 | 16 | 64 |
| CAN-ICU | | | | | | | | | | |
| 61589 | | | | | | | | | | |
| E. faecalis | ND | ND | ND | ND | ND | ND | ND | ND | ND | 256 |
| ATCC29212 | | | | | | | | | | |
| E. facium | ND | ND | ND | ND | ND | ND | ND | ND | ND | 256 |
| ATCC27270 | | | | | | | | | | |
| S. pneumoniae | 4 | 32 | 64 | 128 | 64 | 64 | 128 | >512 | >256 | >128 |
| ATCC49619 | | | | | | | | | | |
| E. coli | 1 | 4 | 16 | 64 | 32 | 128 | 128 | 32 | 32 | 128 |
| ATCC25922 | | | | | | | | | | |
| E. coli | 128 | 8 | 16 | 64 | 64 | 128 | 64 | 64 | 64 | 256 |
| CAN-ICU | | | | | | | | | | |
| 61714 | | | | | | | | | | |
| E. coli | 8 | ND | 64 | 128 | ND | 64 | 128 | 64 | 64 | 256 |
| CAN-ICU | | | | | | | | | | |
| 63074 | | | | | | | | | | |
| P. aeruginosa | 8 | 512 | >512 | 256 | 128 | 128 | 256 | >512 | >256 | 256 |
| ATCC27853 | | | | | | | | | | |
| P. aeruginosa | 128 | 512 | >512 | 256 | 128 | 64 | 128 | 256 | 128 | 256 |
| CAN-ICU | | | | | | | | | | |
| 62308 | | | | | | | | | | |
| hemolytic | <0.6% | <0.3% | ND | ND | 56% | 82% | ND | ND | ND | ND |
| activity at | | | | | | | | | | |
| 100 mg/L | | | | | | | | | | |

| Control | | | | | | | | | |
|---|---|---|---|---|---|---|---|---|---|
| Organism | 9 | 10 | 11 | 12 | 13 | 14 | Kanamycin A | 15 | 16 |
| S. aureus | 4 | 4 | 32 | 8 | 8 | 16 | 4 | 8 | 16 |
| ATCC29213 | | | | | | | | | |
| MRSA | 8 | 4 | 32 | 16 | 8 | 16 | >512 | 16 | 8 |
| ATCC33592 | | | | | | | | | |
| S. epidermidis | 2 | 1 | 4 | 4 | 8 | 4 | 2 | 2 | 8 |
| ATCC14990 | | | | | | | | | |
| MRSE | 2 | 4 | 4 | 8 | 8 | 8 | 128 | 2 | 8 |
| CAN-ICU | | | | | | | | | |
| 61589 | | | | | | | | | |
| E. faecalis | 32 | 4 | 32 | 64 | 32 | 32 | ND | 8 | 16 |
| ATCC29212 | | | | | | | | | |
| E. facium | 16 | 0.5 | 2 | 64 | 16 | 16 | ND | 8 | 16 |
| ATCC27270 | | | | | | | | | |
| S. pneumoniae | 64 | 32 | 64 | 128 | 64 | 64 | 8 | 64 | 128 |
| ATCC49619 | | | | | | | | | |
| E. coli | 16 | 32 | 64 | 64 | 32 | 64 | 8 | 32 | 64 |
| ATCC25922 | | | | | | | | | |
| E. coli | 32 | 64 | 128 | 128 | 32 | 64 | 16 | 32 | 128 |
| CAN-ICU | | | | | | | | | |
| 61714 | | | | | | | | | |
| E. coli | 16 | 32 | 128 | 64 | 32 | 32 | 32 | 32 | 64 |
| CAN-ICU | | | | | | | | | |
| 63074 | | | | | | | | | |
| P. aeruginosa | 32 | 32 | 128 | 128 | 32 | 128 | >512 | 64 | 256 |
| ATCC27853 | | | | | | | | | |
| P. aeruginosa | 32 | 8 | 64 | 64 | 16 | 32 | >512 | 64 | 64 |
| CAN-ICU | | | | | | | | | |
| 62308 | | | | | | | | | |
| hemolytic | 2% | 13% | 82% | 1% | 3% | 2% | <0.4% | 20% | 6.7% |
| activity at | | | | | | | | | |
| 100 mg/L | | | | | | | | | |

ND = not determined

Neomycin B- and kanamycin A-based polyamines exhibit Gram-positive activity and restore antibacterial activity in these two aminoglycosides against MRSA. It appears that conjugation of neomycin B to saturated, aliphatic and long fatty acid chains (>C₁₂) results in optimal Gram-positive activity. Similar trends are observed with polyguanidinylated neomycin B-lipids 8-14. This is in contrast to the antibacterial activities against Gram-negatives such as E. coli. For instance, polyamine-based lipid 1 bearing a C₆-lipid chain displays stronger activity against E. coli than lipids 3 and 4 bearing C₁₆- or C₂₀-tail. Interestingly, conversion of the polyamine-based head group in 2, 3 and 6 into polyguanidino compounds 9, 10 and 13 enhances the antibacterial activity against two P. aeruginosa strains. Of special interest is the high activity of guanidinylated neomycin B-derived lipids 10 (MIC=8) and 13 (MIC=16) against a gentamicin-resistant (as well as neomycin B and kanamycin A-resistant) P. aeruginosa strain. In comparison, kanamycin A-derived cationic C₁₆-lipids 15 and 16 exhibit slightly reduced antibacterial activity when compared to their neomycin B congeners 3 and 10 indicating that the number of cationic charges as well as the nature and size of the polycationic headgroup affects the antibacterial potency. Of special note is the observation that guanidinylated neomycin B C₁₂-lipid 9 results in enhanced antibacterial activity when compared to amine-based neomycin B C₁₂-lipid 2 indicating that guanidinylated neomycin B lipids require a reduced hydrophobic threshold in order to increase the antibacterial effect. Another interesting observation is the significantly improved MRSA activity of guanidinylated neomycin B-pyrene analogs 13 (32-fold lower MIC) and 14 (8-fold lower MIC) when compared to neomycin B-pyrene analogs 6 and 7. This suggests that lipid tails consisting of polyaromatics or steroids in combination with polyguanidinylated neomycin B headgroups are able to confer improved activity against MRSA. Overall the most potent antibacterial agent appears to be guanidinylated neomycin B-C₁₆-lipid 10 which exhibits potent Gram-positive activity while at the same time showing activity against some Gram-negative strains such as E. coli and P. aeruginosa. Moreover, the results indicates that polyguanidinylation of polycationic lipids provides a tool to reduce the hemolytic activity in this class of compounds. For instance, the polyguanidinylated-lipids 10 and 17 exhibit reduced hemolytic activity when compared to their polyamine analogs 3 and 15. This suggests that structural modifications of the polycationic headgroup in aminoglycoside-derived cationic amphiphiles can reduce in vitro toxicity.

In summary, conjugation of neomycin B- and kanamycin A-derived polyamine and polyguanidinylated headgroups to hydrophobic lipid tails restore MRSA activity in both aminoglycosides and MRSE activity in kanamycin A. Optimal Gram-positive activity is achieved by conjugation of the polyamine-based headgroup to saturated C₁₆- or C₂₀-lipid tails. In the case of neomycin B-derived polyguanidinylated headgroups, induction of Gram-positive activity can be achieved by conjugation to shorter C₁₂-lipid tails. Moreover, guanidinylation of the polycationic headgroup in neomycin B-derived cationic lipids enhances antibacterial activity against neomycin B-, kanamycin A- and gentamicin-resistant P. aeruginosa strains as well as MRSA. Furthermore, polyguanidinylated aminoglycoside-based lipids display reduced hemolytic activity when compared to their polyamine analogs.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

Baker et al., J. Org. Chem., 65:9054-58, 2000.
Bera et al., J. Med. Chem., 51:6160-64, 2008.
Dathe et al., Biochemistry, 35:12612-22, 1996.
Dzidic et al., Food Technol Biotech., 46:11-21, 2008.
Gilbert and Moore, J. Appl. Microbiol., 99:703-15, 2005.
Goodman & Gilman's “The Pharmacological Basis of Therapeuticsm
Gordon et al., Contact Dermatitis, 30(3):181-2, 1994.
Greene and Wuts, In: Protective Groups in Organic Synthesis, 2^{nd} Ed.; Wiley, N.Y., 1999.
Haddad et al., Cytokine, 13(3):138-47, 2001.
Hancock and Sahl, Nat. Biotechnol., 24:1551-57, 2006.
Hooper, Antimicrob Agents Chemother., 22(4):662-71, 1982.
Kudyba et al., Carbohydr. Res., 342(3-4):499-519, 2007.
Kügler et al., Microbiology, 151:1341-48, 2005.
Makovitski et al., Proc. Natl. Acad. Sci. USA, 103:15997-6002, 2006.
Physicians Desk Reference
Remington's Pharmaceutical Sciences, 18th Ed. Mack Printing Company, 1990.
Scott et al., Curr. Opinion Biotechnol., 19:620-27, 2008.
Sitaram and Nagaraj, Curr. Drug Targets, 3(3):259-67, 2002.
Smith and March, In: March's Advanced Organic Chemistry: Reactions, Mechanisms, and Structure (March's Advanced Organic Chemistry), 2001.
The Merck Index, Eleventh Edition
Zhanel et al., Antimicrob. Agents Chemother., 52:1430-1437, 2008.

1-42. (canceled)

43. A guanadinylated-aminoglycoside-lipid conjugate comprising
(a) an aminoglycoside group;
(b) at least one guanidino group attached to a primary or secondary carbon atom of the aminoglycoside group; and
(c) at least one lipid group attached through a bond or a linker to a branched carbon atom of the aminoglycoside;
or a salt thereof.

44. The aminoglycoside-lipid conjugate of claim 43, wherein the aminoglycoside-lipid conjugate comprises at least two guanidino groups attached to a primary or secondary carbon atom of the aminoglycoside group.

45. The aminoglycoside-hydrophobe conjugate of claim 43, wherein the linker is an amide linker, an aminoacyl linker, an ester linker, an oxyacyl linker, a thioester linker, an oxythioacyl linker, a carbamate linker, a urea linker, a thiourea linker, an ether linker, a carbonate linker, a triazole linker or an amino linker.

46. The aminoglycoside-lipid conjugate of claim 43, wherein the aminoglycoside is further defined as an aminoglycoside antibiotic.

47. The aminoglycoside-lipid conjugate of claim 46, wherein the aminoglycoside antibiotic is selected from the group consisting of a neomycin, a kanamycin, paromomycin, amikacin, a gentamicin, netilmycin, a streptomycin, tobramycin, a hygromycin, sisomycin and a spectinomycin.

48. The aminoglycoside-lipid conjugate if claim 47, wherein the aminoglycoside antibiotic is neomycin or kanamycin.

49. The aminoglycoside-lipid conjugate of claim 43, wherein the lipid group is alkyl_{(C5-45)} or alkenyl_{(C5-45)}.

50. The aminoglycoside-lipid conjugate of claim 49, wherein the lipid is further defined as —C₅H₁₁, —C₁₁H₂₃, —C₁₅H₃₁, —C₁₉H₃₉, or —C₁₇H₃₁.

51. The aminoglycoside-lipid conjugate of claim 43, wherein the lipid group is cyclic or aromatic.

52. The aminoglycoside-lipid conjugate of claim 51, wherein the lipid group is:

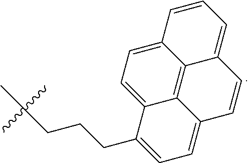 [SMILES: *CCCc1ccc2ccc3cccc4ccc1c2c34]

53. The aminoglycoside-lipid conjugate of claim 43, wherein the aminoglycoside-lipid is present as a trifluoroacetic acid salt.

54. The aminoglycoside-lipid conjugate of claim 43, further defined as: 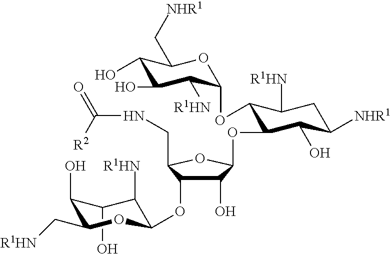
wherein
R₁═C(NH)NH₂; and
R₂═C₅H₁₁, C₁₁H₂₃, C₁₅H₃₁, C₁₉H₃₉, C₁₇H₃₁, 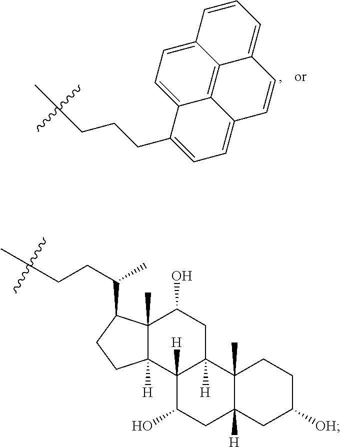
or a salt thereof.

55. The aminoglycoside-lipid conjugate of claim 43, further defined as: 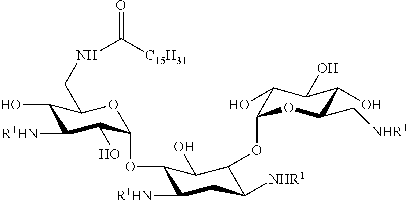
wherein R₁═C(NH)NH₂.

56. A pharmaceutical composition comprising an aminoglycoside-lipid conjugate comprising at least one guanidino group attached to a primary or secondary carbon atom of the aminoglycoside in a pharmaceutically acceptable formulation.

57. The pharmaceutical composition of claim 56, wherein the aminoglycoside-lipid conjugate is further defined as the aminoglycoside-lipid conjugate of claim 43.

58. A method of treating a bacterial infection in a subject comprising administering to the subject an effective amount of an aminoglycoside antibiotic-lipid conjugate comprising at least one guanidino group attached to a primary or secondary carbon atom of the aminoglycoside.

59. The method of claim 58, wherein the aminoglycoside antibiotic-lipid conjugate is further defined as the aminoglycoside-lipid conjugate of claim 43.

60-84. (canceled)
